(19) United States
(12) Patent Application Publication
(10) Pub. No.: US 2007/0212282 A1
(43) Pub. Date: Sep. 13, 2007

(54) Sterilization apparatus using sterilizing superheated steam under normal pressure and hypoxia environment

(75) Inventors: Akira Matsui, Kyoto (JP); Sadanobu Yasu, Yasu-shi (JP); Masaaki Kaneko, Tokyo (JP); Katsuki Mizota, Kitasaitama-gun (JP); Takahiro Hoshino, Gotenba-shi (JP); Tomoichi Shoda, Tokyo (JP)

Correspondence Address: KODA & ANDROLIA, 2029 CENTURY PARK EAST, SUITE 1140, LOS ANGELES, CA, 90067, US

(73) Assignees: Akira Matsui; G C Corporation

(21) Appl. No.: 11/645,944

(22) Filed: Dec. 27, 2006

(30) Foreign Application Priority Data
Dec. 28, 2005 (JP) 2005-378730
Dec. 19, 2006 (JP) 2006-341418

(51) Int. Cl. A61L 2/07

(52) U.S. Cl. 422/298

(57) ABSTRACT

The apparatus comprises a containing portion in which an object to be sterilized such as medical treatment tools is accommodated for sterilization; and a sterilizing superheated steam generating and supplying portion provided adjacent to the containing portion comprising a saturated steam generating portion for generating a saturated steam at 80 to 100 degrees centigrade from water, a sterilizing superheated steam generating portion for generating therein a sterilizing superheated steam at 120 to 300 degrees centigrade by colliding the saturated steam generated from the saturated steam generating portion with a heated superheating medium at a predetermined flow rate; and a sterilizing superheated steam supplying portion for feeding the sterilizing superheated steam into the containing portion, thereby forming in the inner of the containing portion heated low-oxygen environmental condition equal to or less than 3% oxygen concentration suitable for sterilizing attached bacteria, at 120 to 150 degrees centigrade under normal pressure.

### FIELD OF THE INVENTION

The present invention relates to a sterilization apparatus using sterilizing superheated steam generated by colliding the saturated stream with a heated superheated medium under specified condition, more particularly relates to a sterilization apparatus for accommodating and sterilizing therein an object attached with bacteria such as treatment tools for medical or dental use.

In detail, the present invention relates to a sterilization apparatus for sterilizing attached bacteria using a sterilizing superheated steam including steam particle of ultra-fine level (for example, particle diameter is from 1 micron to 400 nanometer) such as unimolecule or plural molecule (for example, molecule number 1 to 10) of H₂O at 120 to 150° C. (degrees centigrade) under normal pressure, the sterilizing superheated steam being generated by colliding a saturated steam at 80 to 100° C. with the surface of heated medium.

The object attached with bacteria, which is to be sterilize should be widely understood in the present invention, for example, treatment tools and used materials for use in medical treatment, dental treatment, physical therapy (chiropractic), veterinary treatment, beauty industry, and barber industry, and medical waste and hazardous waste used in these fields. In the present invention, the object attached with bacteria is sometimes referred as “object to be sterilized” or “treatment tools”.

The term “sterilization” in this specification means to kill and remove all the microorganism different from the terms like pasteurization, disinfection, sanitalization, decontamination, and antibacterial.

### PRIOR ART

The above-mentioned term “sterilization” indicates “killing or removing all the microorganism contained in substance” as defined in the Disinfection and Sterilization Methods of the Japanese Pharmacopoeia (General Test).

In other words, in the filed of sterilization, SAL (Sterility Assurance Level) is defined such that the probability of living microorganisms after effective sterilization is equal to or less than one millionth.

A high-pressure steam sterilization method (autoclave method) has been generally executed for killing dormant spore or sporule in the prior art.

For example, the prior high-pressure steam sterilization method (autoclave sterilization method) for sterilizing spore-forming bacteria in the group of Bacillus or Clostridium with high heat resistance is executed under the following conditions. Namely, the method is executed under the pressure from 1.2 to 1.5 kgf/cm², at 115 to 118° C. for 30 minutes, at 121 to 124° C. for 15 minutes, at 126 to 129° C. for 10 minutes and at 134° C. for 3 minutes.

A heating cooker for cooking food material with superheated steam has been known as an available technology of superheated steam obtained by further heating a saturated steam (see the patent document 1).

It has been also known to sterilize food, medical raw material, cosmetic raw material and grain using a superheated steam as a heating medium (see the patent document 2).

Further, a steam sterilization apparatus housing a saturated steam generation mechanism has been known, in which a medical material such as an operating gown, bandage, gauze or cotton which are used in a medical institution is passed through steam to be disinfected, pasteurized, sanitalized (see the patent documents 3 and 4).

The steam sterilization apparatus includes a superheated steam generation mechanism, however, the generated superheated steam is not directly applied to the object to be sterilized, but the superheated steam is operated in air, namely under the atmosphere existing oxygen.
[Patent Document 1] JP-A-2004-162936
[Patent Document 2] Japanese Patent No.2637530
[Patent Document 3] JP-A-2002-17825
   [Patent Document 4] JP-A-2004-35090

The actual sterilization conditions of the above-mentioned high-pressure steam sterilization method with an autoclave in the prior art are under the pressure 1.2 to 1.5 kgf/cm² at 115 to 118° C. for 30 minutes, at 121 to 124° C. for 15 minutes, at 126 to 129° C. for 10 minutes and at 134° C. for 3 minutes.

Water is heated to generate a saturated steam in the apparatus sealed in such conditions, the temperature of the steam rises while existing with air, and the pressure thereof becomes high like 1.5-2.5 kgf/cm², thereby achieving sterilization. In other words, the boiling point of water is made 115 to 134° C. by pressurizing at 1.5 to 2.5 kgf/cm². Such steam is not a superheated steam, but a pressurized heated steam. It would have to say a temperature suggestion under high temperature of air and saturated steam.

There has not been proposed a sterilization apparatus for sterilizing medical treatment tools which utilizes a sterilizing superheated steam at a specified temperature area under low-oxygen environmental condition and normal pressure and which is superior in sterile property.

The above-mentioned high-pressure steam sterilization apparatus having a saturated steam generation mechanism for sterilizing medical treatment tools (Patent Documents 3 and 4) is not basically improved from the prior idea in which an object is sterilized by filling a sterilization chamber with a saturated steam and by pressurizing the chamger.

A high-pressure steam sterilizer (autoclave) has been generally used for sterilizing medical treatment tools in these days.

Improvement and modification have been highly requested from a medical scene in its operation, cost, safety, efficiency and time, however, remarkable attempts have not been yet found.

Such an autoclave method proposes that an object to be sterilized is contained in an autoclave pot and is sterilized under a pressurized steam at 1.2 to 1.5 kgf/cm² and 118 to 129° C. for 10 to 30 minutes.

### SUMMARY OF THE INVENTION

The present invention proposes a sterilization apparatus superior in economical efficiency, safety, efficiency, reduction of operation time under normal pressure, hypoxic environment and 120 to 150° C. by using a specific superheated steam superior in sterile property with a view to streamlining the prior autoclave sterilization method for sterilizing medical treatment tools.

The inventors of the present invention have studied a sterilization method of medical treatment tools utilizing a superheated steam. As the result, they have found a sterilizing superheated steam in a specific temperature area can kill microorganism, bacteria, fungus, spore, virus, heat-resistant sporulation bacteria in a Bacillus group or Clostridium group, and the like attached to medical treatment tools under normal pressure and low oxygen concentration, namely it has a superior sterilizing ability, and they have completed the present invention. The specific superheated steam is referred as a sterilizing superheated steam in the present invention.

The inventors have found that when a containing portion for accommodating therein an object to be sterilized is filled with a superheated steam at 120 to 150° C. (for example, 140° C.±10° C.) under normal pressure, namely under the pressure open to the atmosphere, which includes ultra-fine particle of water molecular level in which such superheated steam includes molecular level H₂O whose one steam particle includes 1 to 10 H₂O molecules and partly includes H₂ and O, (such steam may be called as molecular steam), a heated low-oxygen environmental condition suitable for sterilization is formed in the containing portion, whereby all the microorganism accommodated in the containing portion can be killed within 10 minutes, and such experimental knowledge has achieved the present invention.

Further the inventors have found a superheated steam with highly superior sterile ability is obtained when a superheated steam is produced from alcohol containing water such as ethyl alcohol and, based on the founding, they have accomplished the sterilization technology of the present invention under normal pressure.

The present invention relates to a sterilization apparatus using sterilizing superheated steam for sterilizing therein attached bacteria comprising:
(1) a containing portion in which an object to be sterilized such as medical treatment tools is accommodated for sterilization; and
(2) a sterilizing superheated steam generating and supplying portion provided adjacent to the containing portion, wherein
(3) the sterilizing superheated steam generating and supplying portion comprises:
   a) a saturated steam generating portion for generating a saturated steam at 80 to 100 degrees centigrade from water,
   b) a sterilizing superheated steam generating portion for generating therein a sterilizing superheated steam at 120 to 300 degrees centigrade by colliding the saturated steam generated from the saturated steam generating portion with a heated superheating medium; and
   c) a sterilizing superheated steam supplying portion for feeding the sterilizing superheated steam generated from the sterilizing superheated steam generating portion into the containing portion so as to form in the inner of the containing portion low-oxygen environmental condition equal to or less than 3% oxygen concentration at 120 to 150 degrees centigrade under normal pressure.

Further, according to the sterilization apparatus using sterilizing superheated steam under normal pressure and low oxygen concentration of the present invention, the sterilizing superheated steam is generated from a saturated steam produced from alcohol containing water in order to improve the sterile performance.

### EFFECT OF THE INVENTION

According to the sterilization apparatus for medical treatment tools utilizing a sterilizing superheated steam of the present invention, medical treatment tools can be remarkably simply and economically sterilized.

In particular, according to the sterilization apparatus for medical treatment tools utilizing sterilizing superheated steam of the present invention, a sterilization operation is simple and a sterilized object can be taken out in a dry condition relatively soon after sterilization. In addition, Bacillus stearothermophilus which is hardly sterilized can be completely sterilized in a short time other than bacteria, fungus, spore and virus.

Still further, a sterilizing superheated steam is produced from alcohol containing water in the present invention, so that a noticeable sterility assurance level, namely SAL, (equal to or less than one millionth) can be accomplished

### BRIEF DESCRIPTION OF THE DRAWINGS

FIG. 1 is a diagram showing the first embodiment of the sterilization apparatus of the present invention.

FIG. 2 is a diagram showing the second embodiment of the sterilization apparatus of the present invention.

### DETAILED DESCRIPTION OF THE NVENTION

Now, the technical structure of the present invention is described.

The present invention has a most important characteristic in that for sterilizing medical treatment tools, it utilizes a superheated steam with high sterile property at a specific temperature zone (temperature area) from 120 to 150° C. under normal pressure and hypoxic atmosphere, of which particles (from 1 micron to 400 nanometer) include ultra-fine particle of H₂O at molecular level (1.5 to 2.0 angstrom).

It is detailed thereinafter.

The inventors have studied a superheated steam and its sterilization property and found that a superheated steam in the temperature area from 120 to 150° C. (for example, 140±10° C.) has a superior sterile ability for spore-forming bacteria (Bacillus subtilis) which is hardly sterilized as well as microorganism, fungus, spore and virus.

Further, they found when a saturated steam at 80 to 100° C. is randomly collided with a heated surface of a resistance heater having a high-temperature surface, preferably 600 to 700° C. at a desirable flow rate (2 m/sec to 4 m/sec), and a superheated steam at 120 to 300° C. was generated, such superheated steam had a superior sterile ability for an object to be sterilized under a normal pressure and hypoxic atmosphere.

The inventors have assumed the reason such superheated steam generated by colliding has a superior sterile ability as follows.

When a saturated steam at 80 to 100° C. is collided with a heated surface of a resistance heater at 600 to 700° C., a large steam particle of saturated steam is instantly and explosively converted into ultra-fine superheated steam particles of 1.5 to 2.0 angstrom including 1 to 10 H₂O molecules and a just little number of H₂ and O.

The volume of H₂O molecule is assumed to be expanded into 1600 times.

The size of steam particle is equal to or less than 1 micron at 80° C. (called as a white steam), 1 micron to 300 nanometer at 100° C. (called as a blue steam) and 300 to 400 nanometer at 120 to 150° C. (called as a clean steam). The superheated steam of the present invention comprises mainly a clean steam, but it should be considered that these three kinds of steam are included.

Such superheated steam of ultra-fine particle (unimolecular) is superior in permeating and invasion ability into cell, virus, fungus, spore, sporule and the like, thereby obtaining superior sterile property Accordingly, a specific superheated steam suitable for sterilization of the present invention is called as a sterilizing superheated steam.

The above-mentioned superheated steam can be produced from saturated steam generated from water such as a purified water or a tap water according to the present invention.

However, when an alcohol containing saturated steam such as ethyl alcohol is generated to produce a superheated steam, a remarkable sterile ability for an object to be sterilized can be achieved

The inventors assume the reason the superheated steam generated from alcohol containing water has superior sterile ability as follows.

The alcohol component in such water highly helps water molecule (polymolecule structure via hydrogen bond) to make ultra-fine or ultra-minute particles. Such ultra-fine or ultra-minute steam particles obtained by alcohol strongly act on solidification of protein constituting the body of microorganism to achieve a superior sterilization property.

Alcohol component in alcohol containing water may be lower alcohol such as methyl alcohol, ethyl alcohol, propyl alcohol, and the like.

Although the concentration of alcohol in the alcohol containing water may be set desirably in the present invention, it may be extremely low (minute amount) and a sufficient effect can be appeared even at 0.1-5%. It suggests that a different sterilization mechanism is acted other than a general “alcohol disinfection” using an alcohol solution at high concentration of 60-70%. The inventors of the present invention assume the mechanism as follows.

The sterilization apparatus for medical treatment tools using a sterilizing superheated steam according to the present invention is preferably constructed so as to obtain the above-mentioned specific superheated steam.

In the sterilization apparatus of the present invention, hardware structures which are used for several kinds of applied instruments utilizing a well-known superheated steam can be used other than that the structure of sterilizing superheated steam generating portion is designed so as to obtain the above-mentioned specific superheated steam.

The hardware structures of prior instruments using a superheated steam are well known and are not explained here.

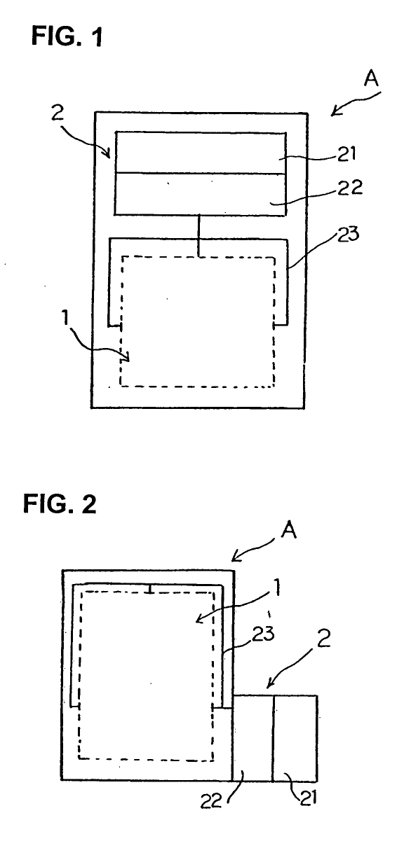

FIG. 1 is a diagram of the sterilization apparatus (A) for medical treatment tools using a sterilizing superheated steam according to the first embodiment of the present invention.

As shown in FIG. 1, the sterilization apparatus (A) of the present invention comprises a containing portion (1) in which medical treatment tools to be sterilized are accommodated and a sterilizing superheated steam generating and supplying portion (2) for generating and feeding the above-mentioned specific sterilizing superheated steam.

The containing portion (1) is provided with an exhaust port for automatically exhausting a superheated steam when a sterilizing superheated steam is filled therein.

According to the sterilization apparatus (A) in FIG. 1, the containing portion (1) and the sterilizing superheated steam generating and supplying portion (2) are integrated so as to form a case.

Although it is not shown in the figure, the sterilizing superheated steam after sterilization treatment may be released outside via a drain so as not to generate secondary contamination or a part of the steam may be reused in view of heat efficiency, if necessary. In case of reuse, it goes without saying that the structure of the sterilization apparatus should be carefully considered in order not to circulate bacteria which are not killed.

The containing portion (1) may be designed so as to facilitate taking in and out of medical treatment tools. A fan or control panel may be provided at desirable portions of the containing portion (1) for controlling drive/stop of the sterilizing superheated stem generating and supplying portion (2), temperature of superheated steam, and spraying amount (exhaust amount) and spraying speed of the superheated steam to the object to be sterilized.

A means may be provided for the containing portion (1) so as not to directly contact the exhaust port (sterilizing superheated steam supplying portion) with the object to be sterilized (medical treatment tools and the like) in order to prevent such damages of high temperature superheated steam supplied or exhausted from the sterilizing superheated steam generating and supplying portion (2) to the object to be sterilized (medical treatment tools) housed in the containing portion (1). If the object to be sterilized (medical treatment tools) is accommodated so as to be 2-3 cm apart from the end of the exhaust port using the means, such damage can be almost prevented.

The sterilizing superheated steam generating and supplying portion (2) may use the hardware of well-known apparatus using superheated steam provided that it can generate a specific superheated steam superior in sterile property.

FIG. 1 diagrammatically shows a saturated steam generating portion (21), a sterilizing superheated steam generating portion (22), a sterilizing superheated steam supplying portion (exhaust port) (23) for feeding and exhausting a sterilizing superheated steam to the containing portion (1), however, the apparatus may be desirably designed by incorporating above-mentioned conditions.

In this invention, a sterilizing superheated steam may be produced from saturated steam generated from alcohol containing water in order to improve the sterile ability of sterilizing superheated steam. In such a case, an alcohol addition mechanism may be provided for the saturated steam generating means (21).

According to the sterilization apparatus of medical treatment tools using a sterilizing superheated steam of the present invention, the method for feeding a specific sterilizing superheated steam to the containing portion (1) from the sterilizing superheated steam generating and supplying portion (2) may be preferably constructed with a fan.

For example, in view of entire and complete sterilization of the object to be sterilized such as medical treatment tools, the sterilizing superheated steam supplying portion (exhaust port) (23) may be designed so as to spray the sterilizing superheated steam from three directions, namely ceiling, a right side and a left side of the containing portion (1) in order that the sterilizing superheated steam at uniform temperature can be sprayed on the entire surface of the medical treatment tools as shown in FIG. 1.

FIG. 2 shows another embodiment of the sterilization apparatus (A) for medical treatment tools using a sterilizing superheated steam according to the present invention.

According to this sterilization apparatus (A), the containing portion (1) and the sterilizing superheated steam generating and supplying portion (2) are separately constructed. The sterilizing superheated steam generating and supplying portion (2) comprises a saturated steam generating portion (21) and a sterilizing superheated steam generating portion (22) as shown in the embodiment 1.

### EMBODIMENT 1

The method for stably producing the sterilizing superheated steam of the present invention was examined at first. The test was executed using a commercially available “Water Oven AX-HC1” which was a household steam cooker using a superheated steam produced by Sharp Corporation. The following embodiment used the AX-HC1.

In the experiment, the menu “Manual steaming, manual heating, wateroven-roast” on the operational panel was used for setting the operational condition of the AX-HC1.

A Chromel-alumel thermo couple of 0.2 mmΦ was provided at 12 places at the center, right and left (3 cm apart from the wall) in the AX-HC1 in advance, thereby measuring the temperature distribution in the AX-HC1. As the result, the sterilizing superheated steam with the temperature distribution from 130 to 140° C. was obtained for 13 minutes at the setting of “wateroven-roast, pre-heated, 130° C., 15 minutes”.

It shows that the sterilizing superheated steam which has been specified in the present invention can be stably produced. The sterilization method and the sterilization apparatus of the present invention have not been known at all as a utilization model (field of application) of the above steam cooker.

### EMBODIMENT 2

A culture bouillon which was inserted with a paper point dipped in saliva of 60 years old male and a culture bouillon to which 0.1 cc of 10-times diluted solution of resting Bacillus subtilis stock which was spore-forming bacteria were prepared at the center of AX-HC1 in order to examine the sterile effect against intraoral indigonous bacteria which was general bacteria and they were sterilized.

The control (comparative group) was, not accommodated in the AX-HC1, prepared in a thermoregulated chamber at 37° C. to be cultivated for a fixed time.

After the experimental group was sterilized while operating the AX-HC1 under the condition that “wateroven-roast, 130° C., 20 minutes”, two experimental bouillon were accommodated in a thermoregulated chamber at 37° C. to be cultivated for a fixed time.

After 24 hours, growth of intraoral indigonous bacteria which was general bacteria was found in the control (comparative group) and CI (Chemical Indicator) resazurin test showed positive, however, the experimental group showed negative.

After 48 hours, breeding was found on the surface of both medium of the control (comparative group), however, the broth madium of the experimental group was transparent and negative.

Accordingly, the above experiment established the sterilization apparatus of the present invention could kill spore-forming bacteria (Bacillus subtilis).

### EMBODIMENT 3

Next, a positive experiment is required to assess whether practical medical treatment tools are sterilized or not by means of the sterilizing superheated steam (120-150° C.) generated under the operational conditions of AX-HC1.

For this purpose, a basic dental treatment set or a dental cutting handpiece was prepared in the above-mentioned position in the AX-HC1 and whether sterilization was effectively executed or not was examined.

A culture bouillon which was inserted with a paper point dipped in saliva of 60 years old male and a culture bouillon to which 0.1 cc of 10-times diluted solution of resting Bacillus subtilis stock, same as the Experiment 2, were accommodated in a sterilization case or a disinfection pack in which a basic treatment set or a dental cutting handpiece was accommodated respectively and the case or pack was prepared in the AX-HC1 to be sterilized.

The control (comparative group) was, not accommodated in the AX-HC1, prepared in a thermoregulated chamber at 37° C. to be cultivated for a fixed time.

The AX-HC1 was operated at the preset condition of “wateroven-roast, 130° C., 30 minutes”, to generate a sterilizing superheated steam at 120-150° C. (130±120−20° C.) under the pressure open to the atmosphere by colliding a saturated steam at 80-100° C. generated from a tap water or a purified water with the heated surface at 600-700° C. of the heater. The sterilizing superheated steam at 120-150° C. (130±120−20° C.) thus generated is fed into the sterilization chamber, as the result low-oxygen environmental condition equal to or lower than 3% oxygen concentration was formed in the sterilization chamber under normal pressure. After sterilization in the sterilization chamber, two experimental bouillon were accommodated in a thermoregulated chamber at 37° C. to be cultivated for a predetermined time.

After 24 hours, growth of intraoral indigonous bacteria was found in the control (comparative group) and CI (Chemical Indicator) resazurin test showed positive, however, the experimental group showed negative.

After 48 hours, breeding was found on the surface of bouillon of the control (comparative group), however, the broth madium of the experimental group was transparent and negative.

<Evaluation of Sterile Effect>

The experimental result is shown in the following Table 1.

In the Table 1 and other tables, (−) shows negative, (+) shows positive, (++) shows strong positive.

The Table 1 shows sterilization result between the required temperature and time for sterilization by sterilizing superheated steam. At the conditions of 100-110° C. for 15 minutes, general bacteria (intraoral indigonous bacteria) shows positive reaction (+) after 24 hours and Bacillus subtilis (spore-forming bacteria) shows a positive reaction (+) after 46 hours. On the other hand, at the conditions at 120-140° C. for 15 minutes, all results show a negative reaction (−). It shows the sterilization method using sterilizing superheated steam of the present invention is superior.

TABLE 1

| | | culture time at 37° C. | | | | | | | | | |
|---|---|---|---|---|---|---|---|---|---|---|---|
| Sterilization | | 16 hours | | 24 hours | | 46 hours | | 67 hours | | 87 hours | |
| condition | | General | BS | General | BS | General | BS | General | BS | General | BS |
| 100° C. | 20 min. | − | − | + | + | − | ++ | + | ++ | + | ++ |
| 110° C. | 20 min. | − | − | − | − | ± | + | − | + | − | + |
| 120° C. | 20 min. | − | − | − | − | − | − | − | − | − | − |
| 130° C. | 20 min. | − | − | − | − | − | − | − | − | − | − |
| 140° C. | 20 min. | − | − | − | − | − | − | − | − | − | − |

| | | | | | | | | | | |
|---|---|---|---|---|---|---|---|---|---|---|
| CONTROL | + | ++ | ++ | + | ++ | ++ | ++ | ++ | ++ | ++ |

General: General bacteria
BS: Bacillus subtilis

The above-mentioned experimental result ascertained that complete sterilization is accomplished if complete sterilization conditions of sterilizing superheated steam such as 120-140° C., 20 minutes (130±10° C.) are arranged under normal pressure and hypoxic atmosphere and proves that the sterilization method using a sterilizing superheated steam according to the present invention is a greatly effective sterilization method.

Accordingly, the present invention provides an ecological, economical, effective, compact and light-weight type sterilization apparatus using a specific sterilizing superheated steam.

### EMBODIMENT 4

ISO (International Organization for Standardization) places resting spore of index bacteria using the following filter paper as carrier as BI (Biological Indicator) as a sterilization biological test in this field on the marked;
(1) In a steam sterilization method, Bacillus steathermophilus 10⁴ (ATTC#7953),
(2) In a dry heat sterilization method, Bacillus subtilis 10⁵ (Bacillus subtilis, ATrC#9372),
(3) In a radiation sterilization method, Bacillus pumilus 10⁵ (ATTC#27142).

The above “AITC” is the abbreviation of American Type Culture Collection” (United States of America).

In the present embodiment, each 8 filter papers of carrier using above (1), (2), (3) and (4) (total 32 sheets) which were dipped in saliva of 60 years old men were accommodated in the AX-HC1, a sterilizing superheated steam was generated from a tap water and they were sterilized at the conditions at 120-140° C. for 20-40minutes and 120-150° C. for 30 minutes.

Then, the carriers of the experimental group (1), (2), (3) and (4) were inserted in a culture broth liquid tube, accommodated in two thermoregulated chambers at 37° C. and 56° C. respectively to be cultivated for a fixed time.

On the other hand, as control (comparative group), the carriers of (1), (2), (3) and (4) were directly inserted in a culture broth liquid tube, arranged in two thermoregulated chambers at 37° C. and 56° C. respectively to be cultivated for a fixed time.

The result is shown in the following Table 2-1 (culture at 37° C.) and Table 2-2 (culture at 56° C).

TABLE 2-1

| | | culture time at 37° C. | | | | | | | | | | | | | | | | | | | |
|---|---|---|---|---|---|---|---|---|---|---|---|---|---|---|---|---|---|---|---|---|---|
| Sterilization | | 16 hours | | | | 24 hours | | | | 46 hours | | | | 67 hours | | | | 87 hours | | | |
| condition | | (1) | (2) | (3) | (4) | (1) | (2) | (3) | (4) | (1) | (2) | (3) | (4) | (1) | (2) | (3) | (4) | (1) | (2) | (3) | (4) |
| 120-140° C. | 20 min. | − | − | − | − | − | − | − | − | − | − | − | − | − | − | − | − | − | − | − | − |
| 120-140° C. | 30 min. | − | − | − | − | − | − | − | − | − | − | − | − | − | − | − | − | − | − | − | − |
| 120-140° C. | 40 min. | − | − | − | − | − | − | − | − | − | − | − | − | − | − | − | − | − | − | − | − |
| 120-150° C. | 30 min. | − | − | − | − | − | − | − | − | − | − | − | − | − | − | − | − | − | − | − | − |

| | | | | | | | | | | | | | | | | | | | | |
|---|---|---|---|---|---|---|---|---|---|---|---|---|---|---|---|---|---|---|---|---|
| CONTROL | − | + | + | + | − | + | + | + | − | + | + | + | − | + | + | + | − | + | + | + |

TABLE 2-2

| | | culture time at 56° C. | | | | | | | | | | | | | | | | | | | |
|---|---|---|---|---|---|---|---|---|---|---|---|---|---|---|---|---|---|---|---|---|---|
| Sterilization | | 16 hours | | | | 24 hours | | | | 46 hours | | | | 67 hours | | | | 87 hours | | | |
| condition | | (1) | (2) | (3) | (4) | (1) | (2) | (3) | (4) | (1) | (2) | (3) | (4) | (1) | (2) | (3) | (4) | (1) | (2) | (3) | (4) |
| 120-140° C. | 20 min. | ± | − | − | − | + | − | − | − | + | − | − | − | + | − | − | − | + | − | − | − |
| 120-140° C. | 30 min. | ± | − | − | − | + | − | − | − | + | − | − | − | + | − | − | − | + | − | − | − |
| 120-140° C. | 40 min. | − | − | − | − | ± | − | − | − | + | − | − | − | + | − | − | − | + | − | − | − |
| 120-150° C. | 30 min. | − | − | − | − | − | − | − | − | − | − | − | − | − | − | − | − | − | − | − | − |

| | | | | | | | | | | | | | | | | | | | | |
|---|---|---|---|---|---|---|---|---|---|---|---|---|---|---|---|---|---|---|---|---|
| CONTROL | + | − | − | − | + | − | − | − | + | − | − | − | + | − | − | − | + | − | − | − |

As shown in Table 2-1 (culture at 37° C.), the experimental groups (1)-(4) shows negative (−) under the sterilization conditions at 120-150° C. for 20-40 minutes.

On the other hand, in the control (comparative group), (1) shows negative (−), but (2)-(4) show positive (+).

As shown in Table 2-2 (culture at 56° C.), the experimental groups (2)-(4) and control (comparative group) (2)-(4) show positive (+).

The experimental group (1) shows positive (+) under the sterilization condition at 120-140° C. for 20-40 minutes, but shows negative (−) under the conditions at 120-150° C. for 30 minutes. In the control (comparative group), (1) shows positive (+) and (2)-(4) shows negative (−). It is because (1) is thormophilic.

Accordingly, it shows that the sterilization conditions should be devised in order to obtain a complete sterile effect for Bacillus steathermophilus which is a thermophilic spore bacteria For example, the temperature for sterilization may be hightened, the process time may be elongated, or the composition ratio of white sterilizing superheated steam, blue one, and transparent one may be controlled.

The present invention has solved it by a simple method of generating a superheated steam from an alcohol containing water as shown in the following embodiment 5, thereby accomplishing complete sterilization.

### EMBODIMENT 5

The experiment in this embodiment was executed under the same conditions of embodiment 4 other than the following.

A sterilizing superheated steam was produced from water containing 1% echylalcohol as the operating conditions of the AX-HC1.

The result is shown in Table 3-1 and Table 3-2.

The Table 3-1 shows the result when the sterilization conditions of the AX-HC1 are 120-140° C. for 20-40 minutes, and 120-150° C. for 25-30 minutes under culture at 37° C.

Table 3-2 shows the result of culture at 56° C. under the same conditions of Table 3-1.

TABLE 3-1

| | | culture time at 37° C. | | | | | | | | | | | | | | | | | | | |
|---|---|---|---|---|---|---|---|---|---|---|---|---|---|---|---|---|---|---|---|---|---|
| Sterilization | | 16 hours | | | | 24 hours | | | | 46 hours | | | | 67 hours | | | | 87 hours | | | |
| condition | | (1) | (2) | (3) | (4) | (1) | (2) | (3) | (4) | (1) | (2) | (3) | (4) | (1) | (2) | (3) | (4) | (1) | (2) | (3) | (4) |
| 120-140° C. | 20 min. | − | − | − | − | − | − | − | − | − | − | − | − | − | − | − | − | − | − | − | − |
| 120-140° C. | 40 min. | − | − | − | − | − | − | − | − | − | − | − | − | − | − | − | − | − | − | − | − |
| 120-150° C. | 25 min. | − | − | − | − | − | − | − | − | − | − | − | − | − | − | − | − | − | − | − | − |
| 120-150° C. | 30 min. | − | − | − | − | − | − | − | − | − | − | − | − | − | − | − | − | − | − | − | − |

| | | | | | | | | | | | | | | | | | | | | |
|---|---|---|---|---|---|---|---|---|---|---|---|---|---|---|---|---|---|---|---|---|
| CONTROL | − | + | + | + | − | + | + | + | − | + | + | + | − | + | + | + | − | + | + | + |

TABLE 3-2

| | | culture time at 56° C. | | | | | | | | | | | | | | | | | | | |
|---|---|---|---|---|---|---|---|---|---|---|---|---|---|---|---|---|---|---|---|---|---|
| Sterilization | | 16 hours | | | | 24 hours | | | | 46 hours | | | | 67 hours | | | | 87 hours | | | |
| condition | | (1) | (2) | (3) | (4) | (1) | (2) | (3) | (4) | (1) | (2) | (3) | (4) | (1) | (2) | (3) | (4) | (1) | (2) | (3) | (4) |
| 120-140° C. | 20 min. | − | − | − | − | − | − | − | − | − | − | − | − | − | − | − | − | − | − | − | − |
| 120-140° C. | 40 min. | − | − | − | − | − | − | − | − | − | − | − | − | − | − | − | − | − | − | − | − |
| 120-150° C. | 25 min. | − | − | − | − | − | − | − | − | − | − | − | − | − | − | − | − | − | − | − | − |
| 120-150° C. | 30 min. | − | − | − | − | − | − | − | − | − | − | − | − | − | − | − | − | − | − | − | − |

| | | | | | | | | | | | | | | | | | | | | |
|---|---|---|---|---|---|---|---|---|---|---|---|---|---|---|---|---|---|---|---|---|
| CONTROL | + | − | − | − | + | − | − | − | + | − | − | − | + | − | − | − | + | − | − | − |

As shown in Table3-1 (culture at 37° C.), the experimental groups (1), (2), (3) and (4) show negative reaction (−) under the conditions of the AX-HC1 at 120-140° C. for 20-40 minutes, and at 120-150° C. for 25-30 minutes.

Further as shown in Table 3-2 (culture at 56° C.), all of them show negative reaction (−) under the conditions of the AX-HC1 at 120-140° C. for 20-40 minutes, and at 120-150° C. for 25-30 minutes.

As mentioned above, an extremely high sterile level (super sterile level) such that the existence probability of microorganism is one millionth (10⁻⁶) under the sterilization conditions at 120-140° C. for 20-40 minutes using a sterilizing superheated steam generated from ethylalcohol containing water, namely a complete sterilization at SAL (Sterility Assurance Level) is achieved.

1. Sterilization apparatus using sterilizing superheated steam for sterilizing therein attached bacteria comprising:
(1) a containing portion in which an object to be sterilized such as medical treatment tools is accommodated for sterilization; and
(2) a sterilizing superheated steam generating and supplying portion provided adjacent to said containing portion, wherein
(3) said sterilizing superheated steam generating and supplying portion comprises:
a) a saturated steam generating portion for generating a saturated steam at 80 to 100 degrees centigrade from water,
b) a sterilizing superheated steam generating portion for generating therein a sterilizing superheated steam at 120 to 300 degrees centigrade by colliding said saturated steam generated from said saturated steam generating portion with a heated superheating medium; and
c) a sterilizing superheated steam supplying portion for feeding said sterilizing superheated steam generated from said sterilizing superheated steam generating portion into said containing portion so as to form in the inner of said containing portion low-oxygen environmental condition equal to or less than 3% oxygen concentration at 120 to 150 degrees centigrade under normal pressure.

2. The sterilization apparatus using sterilizing superheated steam as set forth in claim 1, wherein said saturated steam generating portion generates therein a saturated steam from alcohol containing water.

3. The sterilization apparatus using sterilizing superheated steam as set forth in claim 1, wherein in said sterilizing superheated steam generating and supplying portion, a saturated steam at 80 to 100 degrees centigrade is generated by passing a sprayed atomized water through a boiler, then a superheated steam at 120 to 300 degrees centigrade is generated by colliding said saturated steam with an electric resistance heater with a high temperature surface at 600 to 700 degrees centigrade, while applying blow for acceleration, and said superheated steam thus generated is further fed into said containing portion, whereby said containing portion is filled with said sterilizing superheated steam at 120 to 150 degrees centigrade thereby forming in said inner of said containing portion low-oxygen environmental condition equal to or less than 3% oxygen concentration under normal pressure.

4. The sterilization apparatus using sterilizing superheated steam as set forth in claim 1, wherein said sterilizing superheated steam generating and supplying portion generates a saturated steam at 80 to 100 degrees centigrade and then generates a sterilizing superheated steam at 120 to 300 degrees centigrade in which steam particle including 1 to 10 molecules of H₂O is contained, by colliding said saturated steam with a heated surface of high temperature heater at a predetermined flow rate.

5. The sterilization apparatus using sterilizing superheated steam as set forth in claim 1, wherein said sterilizing superheated steam generating and supplying portion has an exhaust port for spraying a sterilizing superheated steam into said containing portion in which an object to be sterilized is accommodated, and feeds a sterilizing superheated steam at 120 to 150 degrees centigrade into said containing portion so as to fill the inner of said containing portion with it due to the spraying speed and spraying amount of said sterilizing superheated steam, thereby forming low-oxygen environmental condition equal to or less than 3% oxygen concentration under normal pressure in said inner of said containing portion

6. The sterilization apparatus using sterilizing superheated steam as set forth in claim 5, wherein a means for preventing direct contact of an object accommodated in said containing portion to be sterilized such as medical treatment tools with the sterilizing superheated steam sprayed from an exhaust port at the end of said exhaust port is further provided in said containing portion.

7. The sterilization apparatus using sterilizing superheated steam as set forth in claim 6, wherein said object such as medical treatment tolls is accommodated away from the end of said exhaust port at the distance of 2 cm to 3 cm.
